(19) United States
(12) Patent Application Publication
(10) Pub. No.: US 2025/0089982 A1
(43) Pub. Date: Mar. 20, 2025

(54) CONTROL DEVICE, ENDOSCOPE SYSTEM, AND POWER CONTROL METHOD FOR ENDOSCOPE SYSTEM

(71) Applicant: OLYMPUS MEDICAL SYSTEMS CORP., Tokyo (JP)

(72) Inventor: Sayuri YAMAMOTO, Tokyo (JP)

(73) Assignee: OLYMPUS MEDICAL SYSTEMS CORP., Tokyo (JP)

(21) Appl. No.: 18/961,832

(22) Filed: Nov. 27, 2024

Related U.S. Application Data
(63) Continuation of application No. PCT/JP2022/022302, filed on Jun. 1, 2022.

(51) Int. Cl. A61B 1/00

(52) U.S. Cl. CPC A61B 1/00006; A61B 1/00078

(57) ABSTRACT

A control device includes at least one processor having hardware. The processor acquires rigidity setting information that is information relating to rigidity setting of a variable rigidity member and, based on the rigidity setting information, sets a target voltage of a variable voltage power supply that applies a voltage to a constant current circuit for flowing a constant current through a heater that heats the variable rigidity member.

### CROSS REFERENCE TO RELATED APPLICATION

This application is a continuation application of PCT/JP2022/022302 filed on Jun. 1, 2022, the entire contents of which are incorporated herein by this reference.

### BACKGROUND OF THE INVENTION

### 1. Field of the Invention

The present disclosure relates to a control device suitable for rigidity control of a shape-memory alloy, an endoscope system, and a power control method for the endoscope system.

### RELATED ART

In the related art, various types of variable rigidity devices are known for changing rigidity of an endoscope insertion portion. One of the variable rigidity devices is known for increasing rigidity by heating a shape-memory alloy (SMA) member with a heater coil. For example, WO 2018/189888 A discloses a configuration in which a shape-memory alloy member (hereinafter referred to as SMA) is formed into a pipe shape and a heater element (heater coil) is disposed coaxially with the SMA pipe.

The heater coil that heats the SMA produces heat when supplied with power. To enable such power supply, there is known a power control device achieved with a combination of a constant voltage power supply and a constant current drive circuit. This power control device utilizes a power supply voltage generated by the constant voltage power supply and enables a flow of a desired current through a load from the constant current drive circuit.

Raising the power supply voltage generated by the constant voltage power supply and increasing an amount of current from the constant current drive circuit to the heater coil make it possible to raise the temperature of the heater coil to a desired level in a short time. However, after the temperature of the heater coil rises to the desired level, a current required for maintaining the temperature becomes small. For this reason, the power consumption in elements of the constant current drive circuit increases as described later, which results in a waste of power.

On the other hand, JP 2007-35938 A discloses a technique of controlling a power supply voltage by detecting a load current. In addition, JP 2008-305978 A discloses a technique of controlling a power supply voltage by monitoring a gate-drain voltage of a constant current control transistor.

### SUMMARY OF THE INVENTION

A control device according to an aspect of the present disclosure includes at least one processor having hardware. The processor acquires rigidity setting information that is information relating to rigidity setting of a variable rigidity member and, based on the rigidity setting information, sets a target voltage of a variable voltage power supply that applies a voltage to a constant current circuit for flowing a constant current through a heater that heats the variable rigidity member.

An endoscope system according to an aspect of the present disclosure includes: an insertion portion configured to be inserted into a subject; a variable rigidity member that is disposed in the insertion portion and changes in rigidity when heated; a heater that is disposed in the insertion portion and heats the variable rigidity member when energized to produce heat; a constant current circuit for flowing a constant current through the heater; a variable voltage power supply that applies a voltage corresponding to a target voltage to the constant current circuit; and at least one processor having hardware, wherein the processor acquires rigidity setting information that is information relating to rigidity setting of the variable rigidity member and, based on the rigidity setting information, sets the target voltage of the variable voltage power supply.

A power control method for an endoscope system according to an aspect of the present disclosure includes: acquiring rigidity setting information that is information relating to rigidity setting of a variable rigidity member; and setting, based on the rigidity setting information, a target voltage of a variable voltage power supply that applies a voltage to a constant current circuit for flowing a constant current through a heater that is disposed in the insertion portion and heats the variable rigidity member when energized to produce heat.

The present disclosure has the effect of enabling a change into optimal power in a short time.

### BRIEF DESCRIPTION OF THE DRAWINGS

FIG. 1 is a view illustrating a configuration of an endoscope system into which a power control device according to a first embodiment of the present disclosure is embedded;

FIG. 2 is a block diagram illustrating configurations of main parts in the power control device according to the first embodiment;

FIG. 3 is a circuit diagram illustrating an example of a specific configuration of the power control device;

FIG. 4 is a view for describing control by a heat control status setting circuit, a drive current setting circuit, and a power supply voltage setting circuit;

FIG. 5 is a graph for describing SMA target temperatures TH and TL, taking an SMA temperature along the abscissa and an SMA rigidity along the ordinate;

FIG. 6 is a graph taking the time along the abscissa and illustrating changes in SMA temperature, heater current (drive current), and heater voltage when states are changed from State S1 to State S4 in order;

FIG. 7 is a graph illustrating a part of a period illustrated in FIG. 6 enlarged;

FIG. 8 is a circuit diagram illustrating a second embodiment; and

FIG. 9 is a circuit diagram illustrating a third embodiment.

### DETAILED DESCRIPTION OF THE PREFERRED EMBODIMENT(S)

Hereinafter, embodiments of the present disclosure will be described in detail with reference to the drawings.

### FIRST EMBODIMENT

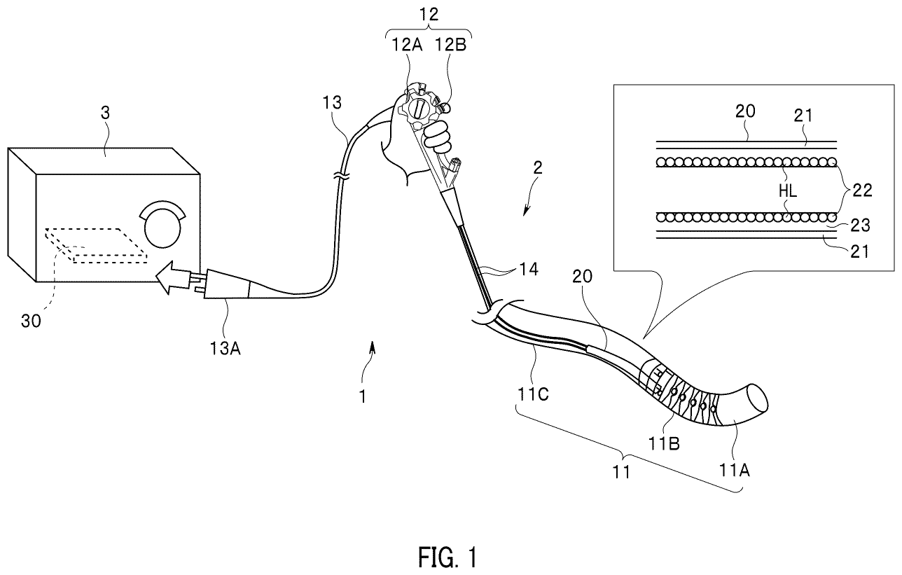

FIG. 1 is a view illustrating a configuration of an endoscope system into which a power control device according to a first embodiment of the present disclosure is embedded. In the present embodiment, a power supply voltage is changed according to a control status, thereby enabling a change into optimal power in a short time. For example, in a case where the present embodiment is applied to power supply for rigidity control of a shape-memory alloy (SMA) in an endoscope system, optimal power can be supplied in a short time by changing a power supply voltage according to a heat control status corresponding to the rigidity of the SMA.

The power control device in the present embodiment is applied to an endoscope system and is applicable not only to an endoscope system but also to various systems in which a load is driven at a constant current.

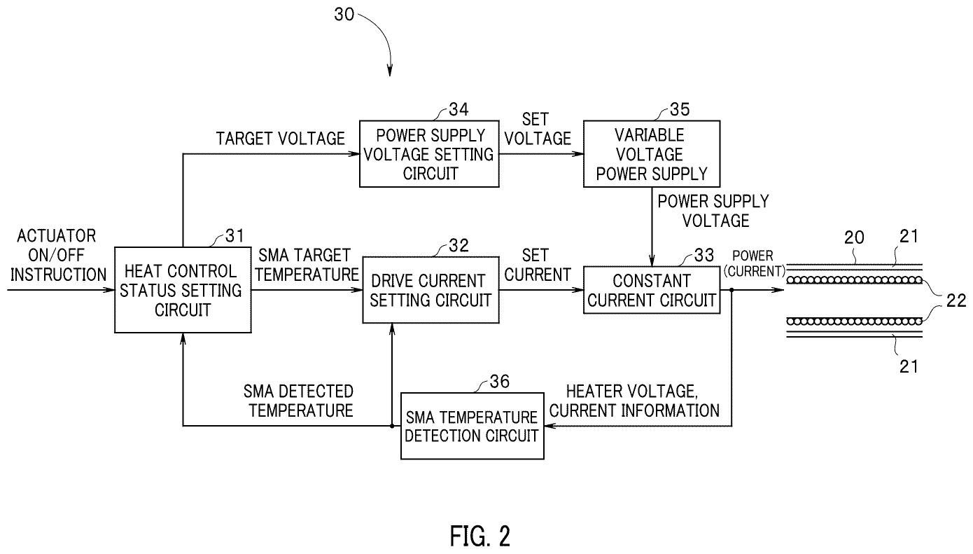

FIG. 1 illustrates a configuration of an endoscope system including the power control device according to the first embodiment of the present disclosure and an endoscope including a variable rigidity device controlled by the power control device. FIG. 2 is a block diagram illustrating configurations of main parts in the power control device according to the first embodiment.

As illustrated in FIG. 1, an endoscope system 1 according to the first embodiment of the present disclosure mainly includes an endoscope 2 inserted into a subject to pick up an endoscopic image of a body cavity, and a processor 3 connected to the endoscope 2 to perform predetermined image processing on the acquired endoscopic image and output the endoscopic image to an external device.

The endoscope 2 includes an insertion portion 11 inserted into the subject, an operation unit 12 provided on the proximal end side of the insertion portion 11, and a universal cord 13 extending from the operation unit 12. The endoscope 2 is detachably connected to the processor 3 through a scope connector 13A provided at an end of the universal cord 13.

The processor 3 in the present embodiment includes a light source device (not illustrated). Furthermore, a light guide (not illustrated) for transmitting illumination light supplied from the light source device and a predetermined electric cable 14 extending from the processor 3 are disposed inside the insertion portion 11, the operation unit 12, and the universal cord 13.

The insertion portion 11 has flexibility and an elongated shape. The insertion portion 11 is also provided with a rigid distal end portion 11A, a bending portion 11B which can be bent freely, and a long flexible tube portion 11C having flexibility in this order from the distal end side.

The distal end portion 11A is provided with an illumination window (not illustrated) for emitting the illumination light transmitted to the subject by the light guide inside the insertion portion 11. The distal end portion 11A is also provided with an image pickup unit (not illustrated) that operates according to an image pickup control signal supplied from the processor 3, picks up an image of the subject illuminated by the illumination light emitted through the illumination window, and outputs an image pickup signal. The image pickup unit includes, for example, an image sensor such as a CMOS image sensor and a CCD image sensor.

The bending portion 11B can be bent according to an operation of an angle knob 12A provided in the operation unit 12.

In the present embodiment, within a variable rigidity range corresponding to a predetermined range from the proximal end portion of the bending portion 11B to the distal end portion of the flexible tube portion 11C, a variable rigidity unit 20 that is configured to change the bending rigidity of the variable rigidity range according to the control of the processor 3 (power control device) is disposed along the longitudinal direction of the insertion portion 11. A specific configuration and the like of the variable rigidity unit 20 will be described later in detail. Hereinafter, for the purpose of illustration, “bending rigidity” is simply abbreviated as “rigidity.”

The operation unit 12 has a shape that is easy for a user to grip and handle. Furthermore, the operation unit 12 is provided with the angle knob 12A that enables operations for bending the bending portion 11B in four directions, that is, up, down, left, and right (UDLR), intersecting the longitudinal axis of the insertion portion 11. The operation unit 12 is also provided with one or more scope switches 12B that follows an instruction according to an input operation from a user.

### <Variable Rigidity Unit 20>

The variable rigidity unit 20 includes a shape-memory alloy (SMA) 21, a heater 22, and a thermal conductive member 23, and the bending rigidity of the variable rigidity range can be changed according to the control of the processor 3 (power control device).

The SMA 21 is a variable rigidity member that has a pipe shape with a small diameter and increases in bending rigidity when heated. In the present embodiment, the SMA 21 is disposed along the longitudinal direction of the insertion portion 11 within the predetermined range from the proximal end portion of the bending portion 11B to the distal end portion of the flexible tube portion 11C in the insertion portion 11 of the endoscope 2. Although the variable rigidity member of the present embodiment has a pipe shape with a small diameter, the variable rigidity member is not limited thereto, and variable rigidity members with various shapes are employable.

The heater 22 includes a heater coil HL disposed in an inside diameter portion of the SMA 21 along the longitudinal direction. The heater coil HL has conductivity. The heater coil HL is formed by a conductor, which is conductive and produces heat when energized by power supply, being wound coaxially with the axis of the SMA 21 and formed into a substantially cylindrical shape.

In the present embodiment, the heater 22 is disposed inside the SMA 21 which is the variable rigidity member, and the outer periphery of the cylindrical coil is disposed along the longitudinal direction while substantially abutting on the inside diameter portion of the SMA 21. The thermal conductive member 23 is disposed between the SMA 21 and the heater 22 and has an effect of reducing a temperature difference between the SMA 21 and the heater 22 including the heater coil HL.

In the present embodiment, the heater 22 is connected to a power control device 30 (to be described) in the processor 3 and produces heat when supplied with power from the power control device 30.

When the endoscope 2 is used, the insertion portion 11 is bent. For this reason, a large force is sometimes applied to the electric cable 14 inside the bending portion 11B, which causes a typical failure mode, that is, a short circuit or disconnection of the electric cable 14. Even when such a failure occurs and the electric cable 14 is short-circuited, constant current drive of the heater 22 makes it difficult to flow a current equal to or higher than a set current, which makes it easy to avoid excessive electric leakage and application of a large voltage. In addition, due to a relatively large length and high resistance of the electric cable 14, the electric cable 14 causes a significant voltage drop, and what is more, the resistance of the electric cable 14 changes depending on temperatures. For these reasons, it is difficult to control supply power with high accuracy by voltage drive.

In terms of safety and power supply accuracy, current drive of the heater 22 is more advantageous than voltage drive of the heater 22 via the electric cable 14. Accordingly, the power control device 30 in the present embodiment includes a circuit achieved by a combination of a variable voltage power supply and a constant current drive circuit and controls the heater 22 with high accuracy by constant current drive.

As described above, heating a shape-memory alloy (SMA) and raising the temperature of the SMA causes a state of high rigidity (hereinafter referred to as rigid state), and lowering the temperature causes a state of low rigidity (hereinafter referred to as flexible state). Therefore, the temperature control of the heater 22 enables rigidity adjustment of the SMA 21 and rigidity control of the variable rigidity unit 20 that includes the SMA 21.

To cause a short-time (quick) rigidity change (phase transformation) in the SMA 21 in order to change states to the rigid state from the flexible state where the temperature is relative low and the rigidity is low in the SMA 21, a large amount of power is supplied to the heater 22. In other words, a sufficiently high power supply voltage is generated in a variable voltage power supply 35 (to be described) of the power control device 30 and supplied to a constant current circuit 33. However, supply power required for the heater 22 changes relatively significantly depending on differences between the temperature to be set in the SMA 21 to control the rigidity of the SMA 21 (hereinafter referred to as SMA target temperature) and the present temperature of the SMA 21. In addition, the resistance value of the heater 22 changes significantly depending on changes in temperature. In other words, while a large amount of power is required for the heater 22 when the SMA 21 transits from the flexible state to the rigid state, the power required for the heater 22 to maintain the rigid state after the SMA 21 enters the rigid state is extremely small as compared with the transition from the flexible state to the rigid state.

That is, to maintain the temperature of the SMA 21 after the temperature of the SMA 21 becomes high and the SMA 21 enters the rigid state, a current flowing through the heater 22 is controlled to be small. However, while the power supply voltage from the variable voltage power supply 35 of the power control device 30 remains relatively high, a decrease of the current flowing through the heater 22 increases the power consumption in a semiconductor device (to be described) at an output stage of the constant current circuit 33 and produces heat.

Therefore, the power control device 30 of the present embodiment utilizes a control status and changes the power supply voltage prior to a possible current change, thereby increasing the power supplied to the heater 22 to a desired level in a short time when the SMA 21 transits from the flexible state to the rigid state and decreasing the power supplied to the heater 22 to a desired level in a short time after the SMA 21 enters the rigid state. That is, the present embodiment enables highly accurate and highly efficient power control while enabling high-speed transition of the variable rigidity unit 20 from the flexible state to the rigid state.

In FIG. 2, the power control device 30 includes a heat control status setting circuit 31, a drive current setting circuit 32, the constant current circuit 33, a power supply voltage setting circuit 34, the variable voltage power supply 35, and an SMA temperature detection circuit 36. Note that the circuits in the power control device 30 excluding the constant current circuit 33 and the variable voltage power supply 35 may be a processor employing a central processing unit (CPU), a field programmable gate array (FPGA), or the like, and may operate according to a program stored in a memory (not illustrated) to control each unit or may implement a part or all of the functions in an electronic circuit of hardware.

An actuator ON instruction to set the rigidity of the variable rigidity unit 20 to the rigid state or an actuator OFF instruction to set the rigidity to the flexible state (hereinafter collectively referred to as actuator ON/OFF instruction when these instructions are not distinguished) is given to the heat control status setting circuit 31. In addition, a presently detected temperature of the SMA 21 (hereinafter referred to as SMA detected temperature) is given to the heat control status setting circuit 31 from the SMA temperature detection circuit 36. The heat control status setting circuit 31 sets a status relating to heat control based on the actuator ON/OFF instruction as rigidity setting information and the SMA detected temperature and sets the SMA target temperature and a target voltage corresponding to the set status. The heat control status setting circuit 31 gives information on the SMA target temperature to the drive current setting circuit 32 and gives information on the target voltage to the power supply voltage setting circuit 34.

The drive current setting circuit 32 also receives the SMA detected temperature input from the SMA temperature detection circuit 36. As described later, the SMA temperature detection circuit 36 receives a voltage across both ends of the heater 22 (hereinafter referred to as heater voltage) and a current flowing through the heater 22 (hereinafter referred to as heater current) and obtains the SMA detected temperature based on the heater voltage and the heater current. The SMA detected temperature indicates the temperature of the heater 22, but a change in temperature of the heater 22 is transmitted to the SMA 21 in an extremely short time. Therefore, the SMA temperature detection circuit 36 uses the detected temperature of the heater 22 as the temperature of the SMA 21 (hereinafter referred to as SMA temperature) and outputs the result of detection, that is, the SMA detected temperature, to the heat control status setting circuit 31 and the drive current setting circuit 32.

Based on a difference between the SMA target temperature and the SMA detected temperature, the drive current setting circuit 32 obtains information on a set current, that is, a set value of the drive current (heater current) that is to flow through the heater 22, and outputs the information to the constant current circuit 33. The drive current setting circuit 32 generates information on the set current such that the larger the difference between the SMA target temperature and the SMA detected temperature the higher the set current becomes and the smaller the difference the lower the set current becomes.

Based on the target voltage, the power supply voltage setting circuit 34 obtains a set voltage, that is, a set value of the voltage to be supplied to the constant current circuit 33, and outputs information on the obtained set voltage to the variable voltage power supply 35. The variable voltage power supply 35 generates a power supply voltage according to the input information on the set voltage and outputs the generated power supply voltage to the constant current circuit 33. The constant current circuit 33 generates a drive current (heater current) to be supplied to the heater 22 using the power supply voltage obtained from the variable voltage power supply 35. The constant current circuit 33 sets an amount of the heater current supplied to the heater 22 based on the set current obtained from the drive current setting circuit 32.

The set voltage is information indicating a voltage value of the power supply voltage to be generated in the variable voltage power supply 35, and the power supply voltage corresponding to the set voltage can be obtained from the variable voltage power supply 35 in a relatively short time after the information on the set voltage is supplied to the variable voltage power supply 35. On the other hand, the set current is information indicating a current value of the drive current to be generated in the constant current circuit 33, and the drive current corresponding to the set current and the power supply voltage can be obtained from the constant current circuit 33 after the information on the set current is supplied to the constant current circuit 33. The temperature of the heater 22 changes at a speed according to the drive current. Therefore, in the present embodiment, instead of changing the power supply voltage by feedback control, the power supply voltage is changed according to a heat control status, whereby the temperature of the heater 22 can reach the SMA target temperature in a relatively short time after an input of the actuator ON instruction to raise the temperature of the heater 22.

A steep rise of the power supply voltage may take time for the voltage to stabilize. For this reason, the power supply voltage setting circuit 34 generates, as the target voltage, a target voltage that rises gradually to the final target voltage at a predetermined tilt.

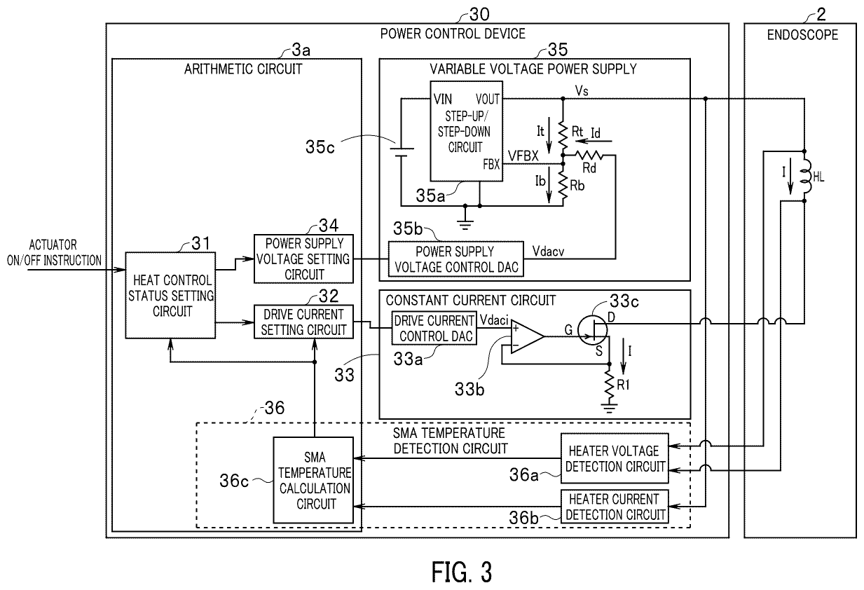

FIG. 3 is a circuit diagram illustrating an example of a specific configuration of the power control device 30.

The power control device 30 in the processor 3 includes the variable voltage power supply 35, the constant current circuit 33, and an arithmetic circuit 3a. The arithmetic circuit 3a may be, for example, an FPGA and includes the heat control status setting circuit 31, the drive current setting circuit 32, the power supply voltage setting circuit 34 of the power control device 30, and an SMA temperature calculation circuit 36c of the SMA temperature detection circuit 36.

The variable voltage power supply 35 includes a step-up/step-down circuit 35a and a power supply voltage control DAC (D/A converter) 35b. A voltage generated by a power supply 35c is supplied to an input terminal VIN of the step-up/step-down circuit 35a. The step-up/step-down circuit 35a steps up or steps down the voltage supplied to the input terminal VIN, generates a power supply voltage Vs, and outputs the power supply voltage Vs from an output terminal VOUT. The output terminal VOUT of the step-up/step-down circuit 35a is connected to a reference potential point via the heater coil HL included in the heater 22, a current path of a transistor 33c included in the constant current circuit 33 (to be described), and a resistor R1.

Resistors Rt and Rb are connected in series between the output terminal VOUT and the reference potential point, and a connection point between the resistors Rt and Rb is connected to a feedback terminal FBX of the step-up/step-down circuit 35a. The connection point between the resistors Rt and Rb is also connected to an output terminal of the power supply voltage control DAC 35b via a resistor Rd.

The power supply voltage control DAC 35b converts information on the set voltage obtained from the power supply voltage setting circuit 34 into an analog signal and outputs a set voltage Vdacv. Assuming that resistance values of the resistors Rt, Rb, and Rd are Rt, Rb, and Rd and that currents flowing through the resistors Rt, Rb, and Rd are It, Ib, and Id, respectively, the step-up/step-down circuit 35a changes the power supply voltage Vs such that a voltage applied to the feedback terminal FBX becomes a specified voltage, that is, Ib (=It+Id) becomes a specified current Ib0. In other words, the step-up/step-down circuit 35a operates to satisfy the following Formula (1).

$$VFBX / Rb = \{ ( Vs - VFBX / Rt \} + \{ ( Vdacv - VFBX ) / Rd \} = Ib 0 \qquad ( 1 )$$

For example, assume that the step-up/step-down circuit 35a steps up the set voltage Vdacv in the state of Ib=Ib0. The current Id increases, and the current Ib is incremented. As a result of this increment, the step-up/step-down circuit 35a lowers the power supply voltage Vs and decrements the current It so as to return the current Ib to the specified current Ib0. In contrast, assume that the step-up/step-down circuit 35a steps down the set voltage Vdacv in the state of Ib=Ib0. The current Id decreases, and the current Ib is decremented. As a result of this decrement, the step-up/step-down circuit 35a raises the power supply voltage Vs and increments the current It so as to return the current Ib to the specified current Ib0. In this manner, the step-up/step-down circuit 35a generates a desired power supply voltage Vs by changing the set voltage Vdacv.

The constant current circuit 33 includes a drive current control DAC 33a, an operational amplifier 33b, and the NMOS transistor 33c. Note that the transistor 33c corresponds to the aforementioned semiconductor device at the output stage of the constant current circuit 33. The drive current control DAC 33a converts the information on the set current obtained from the drive current setting circuit 32 into an analog signal and outputs a control voltage Vdaci corresponding to the set current. The control voltage Vdaci is applied to a positive input terminal of the operational amplifier 33b. An output terminal of the operational amplifier 33b is connected to a gate of the transistor 33c. A drain of the transistor 33c is connected to the output terminal VOUT of the step-up/step-down circuit 35a via the heater coil HL. A source of the transistor 33c is connected to the reference potential point via the resistor R1 and is also connected to a negative input terminal of the operational amplifier 33b.

A voltage at the negative input terminal of the operational amplifier 33b is Vdaci (=I·R1) where I represents a current flowing through the heater coil HL and R1 represents a resistance value of the resistor R1. In other words, with the constant current circuit 33, it is possible to flow a constant current determined by the resistor R1 and the control voltage Vdaci and expressed by the following Formula (2) through the heater coil HL. In a case where the current I is relatively small and the heater voltage is low while the power supply voltage Vs from the step-up/step-down circuit 35a remains relatively high, the power consumption in the transistor 33c increases as described above, resulting in a problem of heat production.

$$I = Vdaci / R 1 \qquad ( 2 )$$

The SMA temperature detection circuit 36 includes a heater voltage detection circuit 36a, a heater current detection circuit 36b, and the SMA temperature calculation circuit 36c. The heater voltage detection circuit 36a is connected to both ends of the heater coil HL included in the heater 22, detects a voltage across the both ends of the heater coil HL (heater voltage), and outputs the result of detection to the SMA temperature calculation circuit 36c. The heater current detection circuit 36b is connected to a wiring between the output terminal VOUT of the step-up/step-down circuit 35a and the heater coil HL, detects a current (heater current) I flowing through the heater coil HL, and outputs the result of detection to the SMA temperature calculation circuit 36c.

The SMA temperature calculation circuit 36c calculates the temperature of the heater coil HL using the results of detection of the heater voltage and the heater current. It should be noted that the heater coil HL and the shape-memory alloy (SMA) 21 have substantially the same temperature. Therefore, as described above, the SMA temperature calculation circuit 36c assumes that the result of calculation, or the temperature of the heater coil HL, matches the temperature of the SMA 21 (hereinafter referred to as SMA temperature) and outputs the SMA temperature as the SMA detected temperature to the heat control status setting circuit 31 and the drive current setting circuit 32.

### (Relation Between Status, Target Temperature, and Target Voltage)

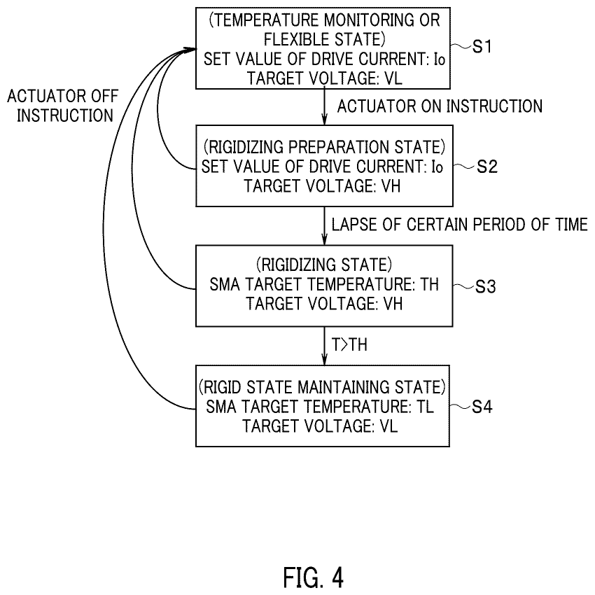

FIG. 4 is a view for describing control by the heat control status setting circuit 31, the drive current setting circuit 32, and the power supply voltage setting circuit 34.

In the example of FIG. 4, four States S1 to S4 are assumed as heat control statuses. State S1 is a state where the actuator OFF instruction is given. State S1 changes to State S2, State S3, and State S4 when the actuator ON instruction is given. When the actuator OFF instruction is given during any of States S2 to S4, the state in process returns to State S1.

State S1 is a state in which the temperature of the SMA 21 is monitored or the SMA 21 is made flexible and the actuator OFF instruction is given. In this case, the heat control status setting circuit 31 instructs the drive current setting circuit 32 to set a relatively low SMA target temperature as the SMA target temperature. Accordingly, the drive current setting circuit 32 sets a set current Io of a relatively low drive current in the constant current circuit 33. In addition, the heat control status setting circuit 31 instructs the power supply voltage setting circuit 34 to set a relatively low target voltage VL. The set current Io is a current set value that causes a relatively small drive current, for example, about 10 mA, to flow through the heater 22.

State S2 is a rigidizing preparation state where the actuator ON instruction is given from State S1. The actuator ON instruction is given at a stage before actually rigidizing the SMA 21. When the actuator ON instruction is given, the heat control status setting circuit 31 instructs the power supply voltage setting circuit 34 to set a relatively high target voltage VH. The target voltage VH is a voltage value that is equal to or higher than a voltage required at the time of rigidizing and is low to the extent possible or equal to an upper limit voltage (a value estimated and set in advance) allowed as a system.

The SMA target temperature obtained from the heat control status setting circuit 31 remains low, and the set current Io obtained by the drive current setting circuit 32 does not change. In other words, the setup performed here is to make the power supply voltage Vs high before increasing the drive current (heater current) of the heater 22. Accordingly, the power supply voltage Vs from the variable voltage power supply 35 rises to the target voltage VH.

State S3 is a state after a lapse of a certain period of time from State S2 and is a stage of actually rigidizing the SMA 21. The heat control status setting circuit 31 instructs the drive current setting circuit 32 to set a relatively high SMA target temperature TH as the SMA target temperature while instructing the power supply voltage setting circuit 34 to set a relatively high target voltage VH. Accordingly, the drive current setting circuit 32 sets a set current of a relatively high drive current in the constant current circuit 33. The constant current circuit 33 increments the heater current flowing through the heater 22. As a result of this increment, the temperature of the SMA 21 rises and the SMA 21 is rigidized.

Although it has been described that a certain period of time elapses when State S2 transits to State S3, this time is a small amount of time and may be, for example, several milliseconds, and State S2 and State S3 may be performed substantially simultaneously. Note that a certain period of time from State S2 to State S3 is a time required for the power supply voltage Vs to rise to a predetermined voltage relatively close to the target voltage VH. In the present embodiment, instead of raising the power supply voltage Vs based on the result of detection of the heater current, the power supply voltage Vs is raised before the heater current is increased or substantially simultaneously with the increase of the heater current. Accordingly, the SMA temperature can reach the SMA target temperature TH in an extremely short time after the actuator ON instruction is given.

State S4 is performed after State S3 and is a state where the rigid state of the SMA 21 is maintained from a state where the SMA detected temperature exceeds the SMA target temperature TH at which the SMA 21 enters the rigid state. Determining that the SMA 21 exceeds the SMA target temperature TH based on the SMA detected temperature obtained from the SMA temperature detection circuit 36, the heat control status setting circuit 31 sets a relatively low target voltage VL in the power supply voltage setting circuit 34 while maintaining the SMA target temperature TL. Note that the target voltage VL is a voltage value (a value estimated and set in advance) that is equal to or higher than a voltage required for maintaining the rigid state and is low to the extent possible. Accordingly, the power supply voltage Vs from the variable voltage power supply 35 decreases to the target voltage VL. The drive current setting circuit 32 generates a set current according to a difference between the SMA detected temperature and the SMA target temperature, and in this case, a relatively low drive current is set. In this way, the heater current is reduced while the temperature of the heater 22 is maintained at the SMA target temperature TL. In this case, the target voltage VL is supplied from the variable voltage power supply 35 to the constant current circuit 33, whereby the power consumed in the transistor 33c of the constant current circuit 33 is reduced.

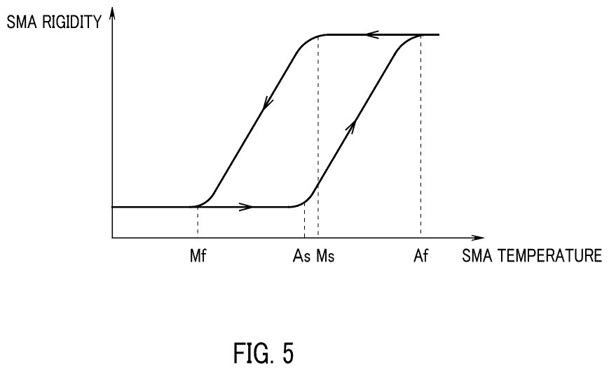

FIG. 5 is a graph for describing SMA target temperatures TH and TL, taking the SMA temperature along the abscissa and the SMA rigidity along the ordinate.

The relation between the SMA temperature and the SMA rigidity is non-linear. The solid line in FIG. 5 indicate hysteresis characteristics of the SMA temperature and the SMA rigidity. The SMA 21 transits to an austenitic phase and has the highest rigidity at a temperature equal to or higher than a predetermined high temperature (hereinafter referred to as austenite transformation completion temperature) Af and transits to a martensitic phase and has the lowest rigidity at a temperature equal to or lower than a predetermined low temperature (hereinafter referred to as martensite transformation completion temperature) Mf.

As illustrated in FIG. 5, in the SMA 21, when the temperature rises from a state of the martensitic phase and the temperature gradually becomes higher than an austenite transformation start temperature As, the rigidity gradually increases and the rigidity becomes the highest at the austenite transformation completion temperature Af or higher. Conversely, in the SMA 21, when the temperature drops from a state of the austenitic phase and the temperature gradually becomes lower than a martensite transformation start temperature Ms, the rigidity gradually decreases and the rigidity becomes the lowest at the martensite transformation completion temperature Mf or lower.

In the present embodiment, for example, the SMA target temperature TH may be set to a predetermined temperature equal to or higher than the austenite transformation completion temperature Af, and the SMA target temperature TL may be set to a temperature equal to or higher than the martensite transformation start temperature Ms and equal to or lower than the SMA target temperature TH, and is lower to the extent possible.

### (Operation)

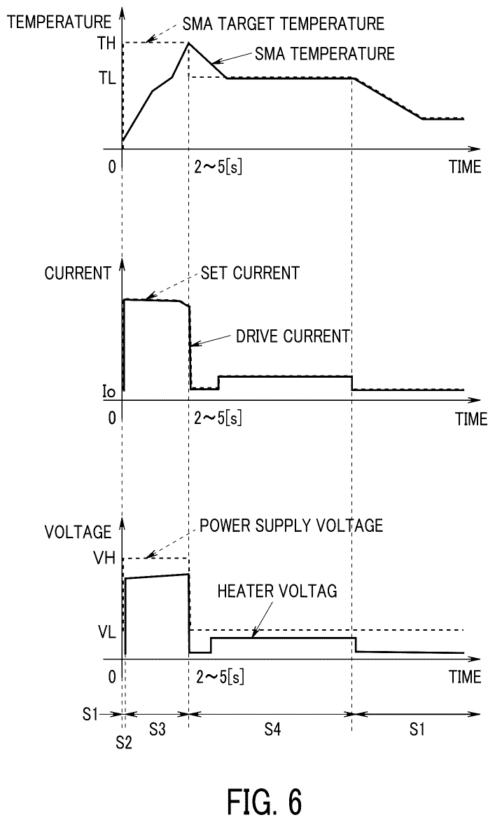

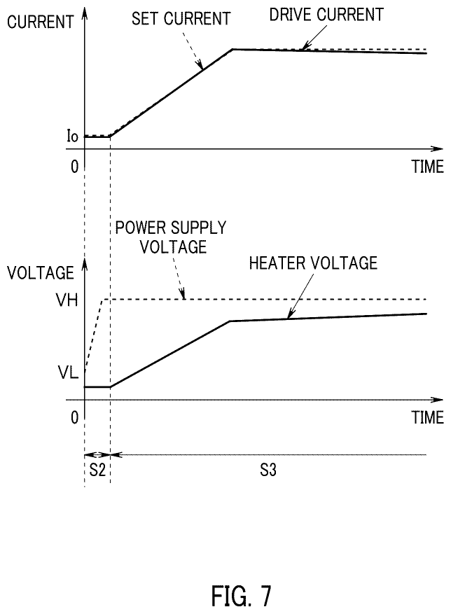

Next, operation of the embodiment having the aforementioned configuration will be described with reference to FIGS. 6 and 7. FIG. 6 is a graph taking the time along the abscissa and illustrating changes in SMA temperature, heater current (drive current), and heater voltage when states are changed from State S1 to State S4 in order. FIG. 7 is a graph illustrating a part of a period illustrated in FIG. 6 enlarged.

Assume that the SMA 21 is in the flexible state where the SMA temperature is lower than the martensite transformation completion temperature Mf and that the power control device 30 is in a state where the set current Io is set at the target voltage VL, that is, in State S1. Time 0 in FIG. 6 indicates State S1. Herein, assume that the actuator ON instruction to increase the rigidity of the SMA 21 is input to the heat control status setting circuit 31. As illustrated in FIG. 6, during the period of State S2, the heat control status setting circuit 31 changes the target voltage VL to the target voltage VH. As indicated by dashed lines in the lower part of FIG. 6 and the lower part of FIG. 7, the power supply voltage Vs from the variable voltage power supply 35 changes from a voltage corresponding to the target voltage VL to a voltage corresponding to the target voltage VH. FIG. 7 illustrates an example in which the target voltage VL becomes closer to the target voltage VH over a predetermined time. Setting the target voltage to VH makes it possible to apply a sufficiently high power supply voltage Vs to the constant current circuit 33.

In State S3 after a lapse of a predetermined time from State S2, the heat control status setting circuit 31 sets the SMA target temperature to the SMA target temperature TH. Accordingly, the drive current setting circuit 32 changes the set current Io to a set current that is based on a difference between the SMA detected temperature and the SMA target temperature TH. The sufficiently high power supply voltage Vs is applied to the constant current circuit 33, and as illustrated in the middle part of FIG. 6 and the upper part of FIG. 7, the drive current (heater current) (solid line) of the heater 22 obtained by the constant current circuit 33 conforms with the set current (dashed line) and increases at a high speed. Accordingly, as illustrated in the upper part of FIG. 6, the SMA temperature (solid line) conforms with the SMA target temperature (dashed line) and reaches the SMA target temperature TH in a relatively short time. In this manner, the SMA 21 transits to a martensitic phase and enters the rigid state.

The SMA temperature of the SMA 21 is detected by the SMA temperature detection circuit 36, and the SMA detected temperature is supplied to the heat control status setting circuit 31. When the SMA temperature reaches the austenite transformation completion temperature Af corresponding to the SMA target temperature TH, the heat control status setting circuit 31 transits to State S4.

In other words, the heat control status setting circuit 31 determines that the SMA 21 is in the rigid state according to the result of detection of the SMA temperature, changes the target voltage VH to the target voltage VL, and changes the SMA target temperature TH to the SMA target temperature TL. In order to reduce the SMA temperature to a temperature corresponding to the SMA target temperature TL in a short time, the power supply voltage setting circuit 34 causes the variable voltage power supply 35 to generate a power supply voltage lower than the target voltage VL, and then, generates a voltage corresponding to the target voltage VL (dashed line in the lower part of FIG. 6). Accordingly, the heater voltage (solid line) changes to a sufficiently low voltage. In addition, after generating a sufficiently low current in the constant current circuit 33, the drive current setting circuit 32 generates a drive current for maintaining the SMA temperature at a temperature as low as possible but higher than the martensite transformation start temperature Ms (the middle part of FIG. 6).

Accordingly, in State S4, the heater 22 is driven by a relatively low heater voltage and a relatively low drive current, and the SMA 21 maintains the rigid state of the martensitic phase. The power supply voltage from the variable voltage power supply 35 is sufficiently low, and the power consumption in the transistor 33c of the constant current circuit 33 is small, which produces less heat.

Next, assume that the SMA 21 is returned from the rigid state to the flexible state. In this case, the actuator OFF instruction to decrease the rigidity of the SMA 21 is input to the heat control status setting circuit 31. Accordingly, the heat control status setting circuit 31 transits to State S1, keeps the target voltage VL, and sets the set current to Io. Accordingly, the drive current setting circuit 32 generates a sufficiently low current in the constant current circuit 33 (the middle part in FIG. 6). In this way, the temperature of the heater 22 decreases, and the SMA temperature becomes lower than the martensite transformation completion temperature Mf, whereby the SMA 21 enters the flexible state.

As described above, the present embodiment enables optimal power supply in a short time by changing a power supply voltage according to a control status. In a case where the present embodiment is applied to power supply for rigidity control of a shape-memory alloy (SMA) of an endoscope system, the rigidity of the shape-memory alloy (SMA) can be changed at high speed. In addition, power is supplied to a heater by a variable voltage power supply and a constant current circuit, so that it is easy to avoid problems such as electric leakage at the time of failure and it is possible to control heat with high accuracy.

### SECOND EMBODIMENT

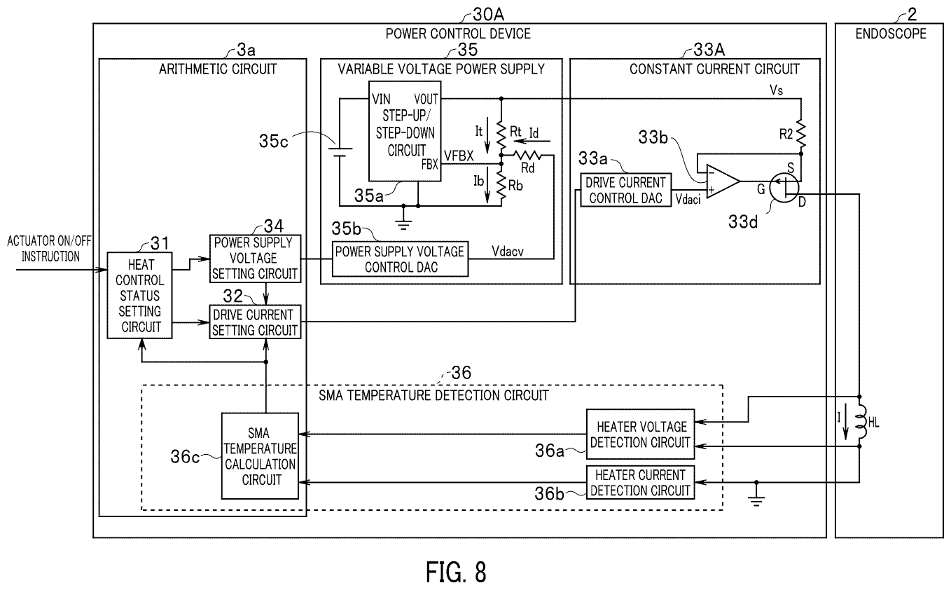

FIG. 8 is a circuit diagram illustrating a second embodiment. FIG. 8 illustrates another example of the specific configuration of the power control device 30. In FIG. 8, the same components as those in FIG. 3 are denoted by the same reference numerals, and description thereof is omitted. The power control device 30 illustrated in FIG. 3 employs the current sink type constant current circuit 33, but a power control device 30A of the present embodiment is different from the first embodiment in that a current source type constant current circuit 33A is employed.

The constant current circuit 33A is different from the constant current circuit 33 in that a PMOS transistor 33d and a resistor R2 are employed instead of the transistor 33c and the resistor R1. In the constant current circuit 33A, an output terminal of an operational amplifier 33b is connected to a gate of the transistor 33d. A source of the transistor 33d is connected to an output terminal VOUT of a step-up/step-down circuit 35a via the resistor R2 and is also connected to a negative input terminal of the operational amplifier 33b. A drain of the transistor 33d is connected to a reference potential point via a heater coil HL.

A voltage at the negative input terminal of the operational amplifier 33b is Vdaci (=I·R2) where I represents a current flowing through the heater coil HL and R2 represents a resistance value of the resistor R2. In other words, with the constant current circuit 33A, it is possible to flow a constant current determined by the resistor R2, a control voltage Vdaci, and a power supply voltage Vs and expressed by the following Formula (3) through the heater coil HL.

$$I = ( Vs - Vdaci ) / R 2 \qquad ( 3 )$$

In order to perform the control expressed by Formula (3), a power supply voltage setting circuit 34 gives information on a set voltage to a drive current setting circuit 32. The drive current setting circuit 32 corrects a set current using the information on the set voltage, and then, outputs the corrected set current to a drive current control DAC 33a.

Other configurations and operation are similar to those in the first embodiment.

Even in the present embodiment, it is possible to obtain similar effects as in the first embodiment.

### THIRD EMBODIMENT

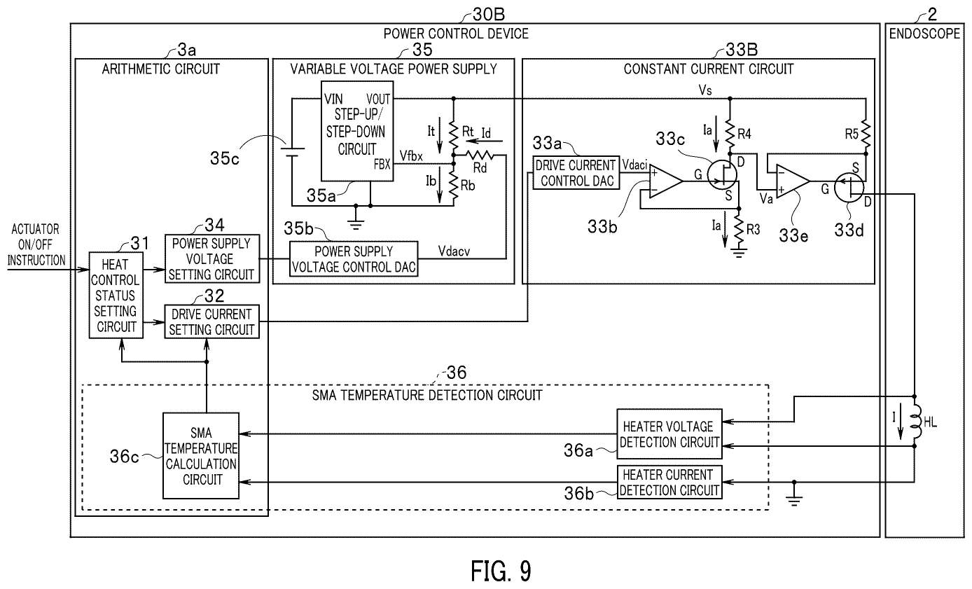

FIG. 9 is a circuit diagram illustrating a third embodiment. FIG. 9 illustrates another example of the specific configuration of the power control device 30. In FIG. 9, the same components as those in FIGS. 3 and 8 are denoted by the same reference numerals, and description thereof is omitted. Formula (3) described above indicates that a heater current is affected by a power supply voltage Vs. In the present embodiment, a current sink type constant current circuit is employed for generating a reference voltage of a current source type constant current circuit. With this configuration, a drive current is prevented from fluctuating due to fluctuation of a power supply voltage, which enables highly accurate current drive. Accordingly, it is possible to set the power supply voltage and the drive current independently, as with the example of FIG. 3.

A power control device 30B in the present embodiment is different from the first and second embodiments in that a constant current circuit 33B is employed. The constant current circuit 33B includes an NMOS transistor 33c, a PMOS transistor 33d, an operational amplifier 33e, and resistors R3 to R5 in addition to a drive current control DAC 33a and an operational amplifier 33b.

An output terminal of the operational amplifier 33b is connected to a gate of the transistor 33c. A drain of the transistor 33c is connected to an output terminal VOUT of a step-up/step-down circuit 35a via the resistor R4. A source of the transistor 33c is connected to a reference potential point via the resistor R3 and is also connected to a negative input terminal of the operational amplifier 33b.

The drain of the transistor 33c is connected to a positive input terminal of the operational amplifier 33e. An output terminal of the operational amplifier 33e is connected to a gate of the transistor 33d. A source of the transistor 33d is connected to the output terminal VOUT of the step-up/step-down circuit 35a via the resistor R5 and is also connected to a negative input terminal of the operational amplifier 33e. A drain of the transistor 33d is connected to the reference potential point via a heater coil HL.

Assume that a current flowing through the heater coil HL is denoted by I, resistance values of the resistors R3 to R5 are denoted by R3 to R5, respectively, and a current flowing through the resistors R3 and R4 is denoted by Ia. A voltage at the negative input terminal of the operational amplifier 33b is Vdaci (=Ia·R3). That is, the current Ia flowing through the resistors R3 and R4 is expressed by the following Formula (4).

$$Ia = Vdaci / R 3 \qquad ( 4 )$$

Therefore, a voltage Va expressed by the following Formula (5) is applied to the positive input terminal of the operational amplifier 33e.

$$Va = Vs - R 4 \cdot Ia = Vs - Vdaci \cdot ( R 4 / R 3 ) \qquad ( 5 )$$

The voltage Va is a source voltage of the transistor 33d, and a current I flowing through the resistor R5, that is, a drive current I, is expressed by the following Formula (6).

$$I = ( Vs - Va ) / 5 = \{ R 4 / ( R 3 \cdot R 5 ) \} Vdaci \qquad ( 6 )$$

Formula (6) indicates that a heater current is determined by a control voltage Vdaci and the resistors R3 to R5. In other words, in the present embodiment, a power supply voltage setting circuit 34 and a drive current setting circuit 32 enable generation of a set voltage and a set current independently of each other.

Even in the present embodiment, it is possible to obtain similar effects as in the first and second embodiments. Furthermore, in the present embodiment, a power supply voltage and a drive current can be set independently. Therefore, the present embodiment enables control with higher degree of freedom and with higher accuracy than the second embodiment.

### (Setting of Upper Limit Value)

Note that a set voltage Vdacv for determining a power supply voltage Vs and a control voltage Vdaci for determining a drive current I may be limited to values in predetermined ranges so as not to set an excessively high power supply voltage Vs and an excessively high drive current Is. That is, the power supply voltage control DAC 35b and the drive current control DAC 33a generate a set voltage Vdacv and a control voltage Vdaci that satisfy the following Formula (7) where Vdacv_clip is a lower limit value of the set voltage Vdacv that defines the upper limit of the power supply voltage Vs and Vdaci_clip is an upper limit value of the control voltage Vdaci that defines the upper limit of the drive current Is.

$$Vdacv > Vdacv\_clip \qquad ( 7 )$$

$$Vdaci < Vdaci\_clip$$

### (Activation Order of Circuit)

At the time of startup or reset, the set voltage Vdacv of the power supply voltage control DAC 35b tends to be 0 [V]. For this reason, there is a possibility that the power supply voltage Vs at an output terminal VOUT of the step-up/step-down circuit 35a becomes extremely high.

Therefore, at the time of startup or reset, first, the power supply voltage control DAC 35b is activated, and after the operation of the power supply voltage control DAC 35b is stabilized and an output satisfying the above Formula (7) is obtained, other circuits such as the step-up/step-down circuit 35a and the constant current circuit 33 (33A, 33B) are activated. In addition, when the power is turned off, the operation of other circuits such as the step-up/step-down circuit 35a and the constant current circuit 33 (33A, 33B) are stopped, and then, the operation of the power supply voltage control DAC 35b is stopped at the end.

At the time of outputting the power supply voltage Vs from the variable voltage power supply 35, an output of the power supply voltage control DAC 35b can be made larger than the lower limit value Vdacv_clip. Accordingly, it is possible to prevent the power supply voltage Vs from exceeding the upper limit voltage or to prevent the drive current from flowing while the power supply voltage Vs exceeds the upper limit voltage. It is also possible to prevent the drive current from exceeding the upper limit current.

The present disclosure is not limited to the embodiments as they are. The present disclosure can be embodied by modifying components without departing from the gist thereof at the stage of implementation. Various disclosures can be made with any appropriate combinations of a plurality of constituent elements disclosed in the above embodiments. For example, some of the constituent elements described in the embodiments may be removed. Furthermore, components in different embodiments may be combined as appropriate.

What is claimed is:

1. A control device comprising at least one processor having hardware,
wherein the processor:
acquires rigidity setting information that is information relating to rigidity setting of a variable rigidity member; and
based on the rigidity setting information, sets a target voltage of a variable voltage power supply that applies a voltage to a constant current circuit for flowing a constant current through a heater that heats the variable rigidity member.

2. The control device according to claim 1,
wherein the processor sets the target voltage independently of a current value of the constant current circuit.

3. The control device according to claim 1,
wherein the processor acquires information on a temperature of the variable rigidity member and sets the target voltage based on the rigidity setting information and the information on the temperature of the variable rigidity member.

4. The control device according to claim 1,
wherein the processor acquires information on a temperature of the variable rigidity member and sets magnitude of a current of the constant current circuit based on the rigidity setting information and the information on the temperature of the variable rigidity member.

5. The control device according to claim 4,
wherein the processor raises the target voltage to a predetermined value or higher and increases a set value of the current of the constant current circuit when the rigidity setting information gives an instruction to increase rigidity and the variable rigidity member has a temperature equal to or lower than the predetermined value.

6. The control device according to claim 4,
wherein the variable rigidity member is a shape-memory alloy, and
the processor:
raises the target voltage to a predetermined value or higher when the rigidity setting information gives an instruction to increase rigidity and a shape-memory alloy has a temperature equal to or lower than the predetermined value; and
increases a set value of the current of the constant current circuit after raising the target voltage to the predetermined value or higher.

7. The control device according to claim 4,
wherein the variable rigidity member is a shape-memory alloy, and
the processor:
raises the target voltage of the variable voltage power supply to a first voltage value when acquiring information to increase rigidity as the rigidity setting information; and
sets magnitude of the current of the constant current circuit to a current value at which the temperature of the variable rigidity member becomes equal to or higher than an austenite transformation completion temperature.

8. The control device according to claim 7,
wherein the processor:
sets the target voltage of the variable voltage power supply to a second voltage value smaller than the first voltage value when acquiring information to maintain rigidity as the rigidity setting information; and
sets magnitude of the current of the constant current circuit to a current value at which the temperature of the variable rigidity member becomes equal to or higher than a martensite transformation start temperature and equal to or lower than the austenite transformation completion temperature.

9. The control device according to claim 7,
wherein the processor sets magnitude of the current of the constant current circuit to a current value at which the temperature of the variable rigidity member becomes equal to or lower than a martensite transformation start temperature when acquiring information to be made flexible as the rigidity setting information.

10. The control device according to claim 1,
wherein the constant current circuit is electrically connected to the heater to form the constant current circuit of a source type.

11. The control device according to claim 10, further comprising:
a constant current circuit of a sink type that generates a reference voltage of the constant current circuit of the source type.

12. The control device according to claim 1,
wherein the processor sets the target voltage such that a voltage applied to the constant current circuit by the variable voltage power supply does not exceed a first upper limit value.

13. The control device according to claim 2,
wherein the processor sets magnitude of a current of the constant current circuit not to exceed a second upper limit value.

14. The control device according to claim 2, further comprising:
a D/A converter that controls power of the variable voltage power supply,
wherein the processor activates the variable voltage power supply after activating the D/A converter.

15. An endoscope system comprising:
an insertion portion configured to be inserted into a subject;
a variable rigidity member that is disposed in the insertion portion and changes in rigidity when heated;
a heater that is disposed in the insertion portion and heats the variable rigidity member when energized to produce heat;
a constant current circuit for flowing a constant current through the heater;
a variable voltage power supply that applies a voltage corresponding to a target voltage to the constant current circuit; and
at least one processor having hardware,
wherein the processor:
acquires rigidity setting information that is information relating to rigidity setting of the variable rigidity member; and
based on the rigidity setting information, sets the target voltage of the variable voltage power supply.

16. The endoscope system according to claim 15,
wherein the processor sets the target voltage independently of a current value of the constant current circuit.

17. The endoscope system according to claim 15,
wherein the processor acquires information on a temperature of the variable rigidity member and sets the target voltage based on the rigidity setting information and the information on the temperature of the variable rigidity member.

18. The endoscope system according to claim 15,
wherein the processor acquires information on a temperature of the variable rigidity member and sets magnitude of a current of the constant current circuit based on the rigidity setting information and the information on the temperature of the variable rigidity member.

19. A power control method for an endoscope system comprising:
acquiring rigidity setting information that is information relating to rigidity setting of a variable rigidity member; and
setting, based on the rigidity setting information, a target voltage of a variable voltage power supply that applies a voltage to a constant current circuit for flowing a constant current through a heater that is disposed in an insertion portion configured to be inserted into a subject and heats the variable rigidity member when energized to produce heat.

20. The power control method according to claim 19,
wherein the target voltage is set independently of a current value of the constant current circuit.
